(19) United States
(12) Patent Application Publication
(10) Pub. No.: US 2014/0214050 A1
(43) Pub. Date: Jul. 31, 2014

(54) GROOMING APPARATUS INCLUDING TICK REMOVER

(71) Applicant: TOPET INTERNATIONAL CO., LIMITED, Kwun Tong (HK)

(72) Inventor: Yueh Yu Wang, Kwun Tong (HK)

(73) Assignee: TOPET INTERNATIONAL CO., LIMITED, Kwun Tong (HK)

(21) Appl. No.: 13/754,652

(22) Filed: Jan. 30, 2013

(51) Int. Cl. A01K 13/00; A61B 17/50

(52) U.S. Cl. CPC A01K 13/00; A61B 17/50
USPC 606/131

(57) ABSTRACT

A grooming apparatus including a tick remover. The tick remover may be a slot having opposing edges for engaging a tick therebetween for removal from the skin of a host. The slot may extend in a direction corresponding to the longitudinal axis of the grooming apparatus.

### BACKGROUND

Ticks and other parasites may become embedded in the skin of a host. Ticks and parasites should be removed from the host as soon as possible because ticks and the like may transmit diseases to the host (e.g., Lyme disease) or the location at which the tick is embedded may become infected. Oftentimes, especially with pet animals, the discovery of ticks occurs during grooming.

Traditionally, ticks have been removed by gripping the body of the tick with tweezers or the like and pulling the embedded portion of the tick from the skin of the host. However, when removing a tick with tweezers, the tick body may be squeezed or severed by the tweezers. In this regard, the tick body may become detached from the head or mouth parts of the tick, which may remain embedded in the host. Without removal the head or mouth parts, which may become more difficult to remove from the host once detached from the body, the still embedded head or mouth parts may become infected or transmit disease to the host. Furthermore, if the body of the tick is squeezed, the tick may regurgitate blood and/or saliva back toward the host, further increasing the risk for infection or transmission of disease associated with the embedded tick.

Because embedded ticks may be difficult to detect, particularly in heavily furred animals such as dogs, the discovery of ticks often occurs during grooming. Discovery of a tick embedded in a host during grooming may interrupt the grooming for at least the time it takes to remove the tick. Additionally, the removal of the tick may require tools other than those used for grooming (e.g., tweezers, etc.). Thus, upon discovery of a tick, the grooming process may also be interrupted while tools for removing the tick are located. In this regard, additional time and effort may be added to grooming. Furthermore, in the event such tools are not available at the time the tick is discovered, the tick may remain embedded in the host for a longer duration, increasing the risk of infection or transmission of disease associated with the embedded tick.

### SUMMARY

In light of the foregoing, grooming apparatuses are described herein that may include a tick remover disposed on a handle portion of the grooming apparatus. As such, when a user discovers an embedded tick on a host during grooming, the tool being used for grooming may additionally be used in the removal of the tick without having to interrupt the grooming process and/or change tools to facilitate removal of the embedded tick. As such, the grooming process may become more efficient, especially for a host having a number of embedded ticks. Furthermore, as the tick remover may be provided on the handle of the grooming apparatus, the operation of the tick remover may be ergonomic and allow a user to remove a tick with reduced effort. Furthermore, the structure of the tick remover may reduce the potential that the body of the tick becomes separated from the head parts of the tick during removal and may reduce the potential of the tick regurgitating blood and/or saliva toward the host upon removal.

A first aspect described herein includes a grooming apparatus. The grooming apparatus may include a handle portion grippingly engageable by a user and having a handle sidewall defining a non-planar cross section. At least a portion of the handle portion may define a longitudinal axis of the grooming apparatus. The grooming apparatus may also have a slot that is defined in the sidewall and that generally extends in a direction corresponding to the longitudinal axis. The slot is configured to engage at least a portion of a tick between opposing edge portions of the slot. In this regard, the size and/or shape of the slot may facilitate engagement of at least a portion of the tick as will become apparent from the description below.

A second aspect described herein includes a grooming apparatus that includes a handle portion having a first end and a second end opposite the first end, a grooming tool at the second end, and a slot extending from the first end. The handle portion may be grippingly engageable by a user and has a handle sidewall defining an arcuate cross section at least at the first end. A longitudinal axis extends along at least a portion of the handle portion between the first end and the second end. The slot is defined in the sidewall and extends from the first end in a direction corresponding to the longitudinal axis and is configured to engage at least a portion of a tick between opposing edges of the slot.

A number of feature refinements and additional features are applicable to the first and second aspects. These feature refinements and additional features may be used individually or in any combination. As such, each of the following features that will be discussed may be, but are not required to be, used with any other feature or combination of features of the first and/or aspects.

For example, in one embodiment, the handle portion may include an arcuate cross section at least along a portion thereof. The arcuate cross section may be elliptical. For example, the arcuate cross section may be substantially circular. In another embodiment, the slot may extend longitudinally from a first end of the handle portion. The opposing edges of the slot may converge as the edges extend away from the first end. Accordingly, as the slot is advanced with respect to the tick, the opposing edges may converge toward the body of the tick until the tick is securely received between the opposing edges. The opposing edges may also be chamfered. For example, the chamfered opposing edges may converge toward an exterior surface of the handle sidewall. As such, the chamfered opposing edges may generally correspond to the shape of the tick body such that the tick body contacts a surface area along the chamfered opposing edges. In this regard, a force applied to the tick to remove the tick may be distributed over a relatively large surface area of the chamfered opposing edges, thus reducing the forces applied to the tick upon removal and reducing the potential for squeezing and/or severing the tick during removal.

In another embodiment, the handle sidewall may define an opening to a hollow space at the first end such that at least a portion of the tick may be received in the hollow space when engaged by the slot. The opposing edges may be rounded adjacent to the first end. Accordingly, the initial engagement of the tick by the slot may be more easily facilitated as the rounded portions of the slot may assist in directing a tick toward and/or into the slot.

In one embodiment, the handle portion may include a recessed portion offset from the slot adjacent to the first end to provide a line of sight to the slot for a user. For example, the recessed portion may be provided on a side of the handle opposite the slot at the first end such that the portion of the handle between the user and the slot may be at least partially recessed. This may allow the user to view the slot or a portion thereof (e.g., the opening of the slot) to assist in engaging a tick with the slot.

In yet another embodiment, the slot may be offset about the handle portion from a direction in which a grooming tool extends from the handle portion. Accordingly, if the handle portion is advanced along the skin of the host in close relation (e.g., contacting relation), the grooming tool may extend from a side of the handle offset from the slot (e.g., by at least about 90° or even 180°) such that the grooming tool does not contact or otherwise interfere with the skin of the host as the slot is advanced with respect to the host.

In one embodiment, the slot may extend longitudinally from the first end a distance not less than about 5 mm. Furthermore, a width of the arcuate cross section may not be less than about 15 mm and may not be greater than about 30 mm. The handle sidewall may not be less than about 1 mm thick and may not be greater than about 10 mm thick. The opposing edges may define an angle therebetween of not more than about 10 degrees. In an embodiment, the grooming apparatus may be constructed from a polymeric material. For example, the polymeric material may be ABS plastic.

In various embodiments, the grooming tool may be selected from the group comprising a pin brush, a bristle brush, a deshedder, a pin comb, a flea comb, a slicker brush, an undercoat rake, a matt breaker, and combinations thereof. The grooming tool may be integrally provided with the handle portion.

A third aspect includes a method for removing an embedded tick from the skin of a host. The method includes advancing a first end of a handle portion along the surface of the skin of the host in which the tick is embedded. The handle portion is advanced in a direction corresponding to a longitudinal axis of the handle portion. The method further includes engaging, in response to the advancing, at least a portion of a tick in a slot defined in a sidewall of the handle portion at the first end. The method also includes moving the slot away from the surface of the skin of the host in a direction generally perpendicular to the longitudinal axis of the tool handle and removing the tick from the skin in response to the moving.

The present invention is directed to the embodiments and aspects that are summarized above, alone or in any combination, as well as additional embodiments and aspects and combinations thereof that will be apparent from the following description of the invention. However, the foregoing summary is intended to provide a basic understanding of at least some embodiments and aspects of the invention. This summary is not an extensive overview of the invention. It is not intended to identify key or critical elements of the invention or to delineate the scope of the invention. The foregoing summary merely presents some concepts of the invention in general form as a prelude to a more detailed description provided below.

### BRIEF DESCRIPTION OF THE FIGURES

FIG. 1 is a perspective view of an embodiment of a tick removal device located at a first end of a handle portion of a grooming apparatus.

FIG. 2 is another view of the embodiment of the tick removal device of FIG. 1.

FIG. 3 is a cross sectional view of a portion of the handle portion when engaged with a tick.

FIG. 4 is a perspective view of another embodiment of a tick removal device.

FIGS. 5-12 depict various embodiments of a grooming apparatus including a tick removal device and a grooming tool.

### DETAILED DESCRIPTION

The following description is not intended to limit the invention to the forms disclosed herein. Consequently, variations and modifications commensurate with the following teachings, skill and knowledge of the relevant art, are within the scope of the present invention. The embodiments described herein are further intended to explain modes known of practicing the invention and to enable others skilled in the art to utilize the invention in such, or other embodiments and with various modifications required by the particular applications(s) or use(s) of the present invention.

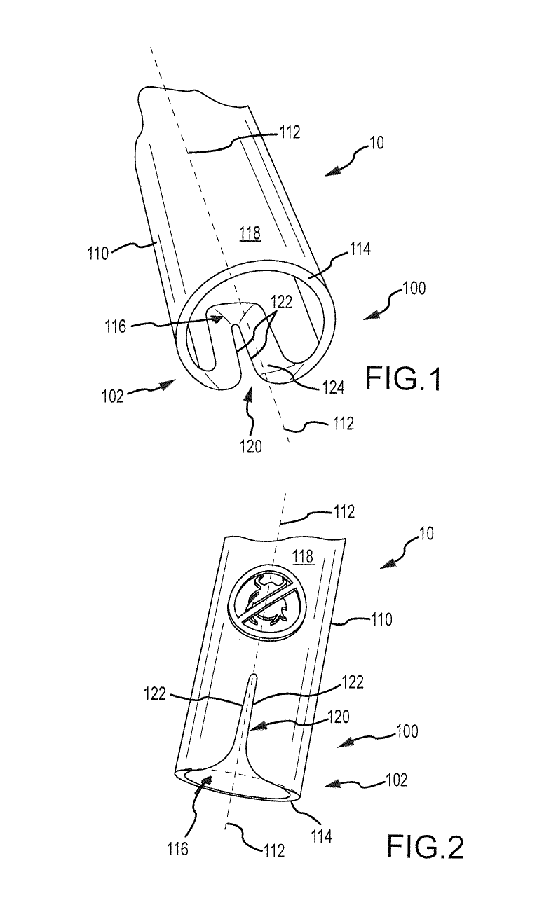

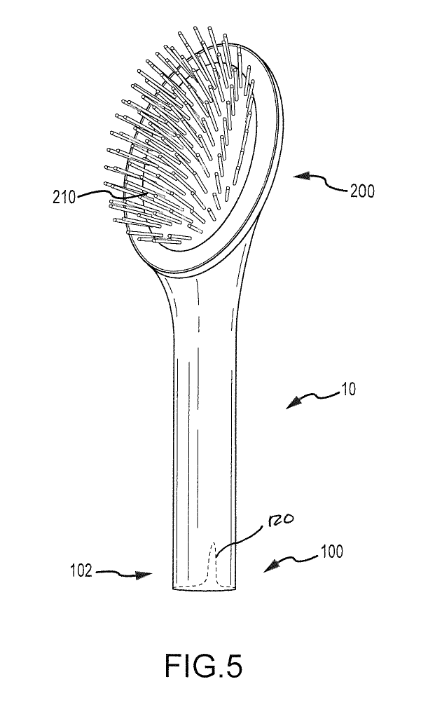

FIGS. 1 and 2 depict an embodiment of a tick remover 100 provided on a grooming apparatus 10. For example, the tick remover 100 may be provided on a handle 110 of the grooming apparatus 10. Accordingly, in some embodiments a grooming apparatus 10 may include the tick remover 100 in combination with a grooming tool as will be described in greater detail below with respect to FIGS. 5-12. In any regard, at least a portion the handle 110 may extend along at least a portion of a longitudinal axis 112. In this regard, the longitudinal axis 112 may correspond with a major dimension (e.g., a length) of the handle 110. For example, in one embodiment, a majority of the length of the handle 110 may extend along the longitudinal axis. Furthermore, as depicted in FIG. 13, a portion of the handle 110 may extend along the longitudinal axis 112 and a portion of the handle 110 may be offset from the longitudinal axis 112. As can be appreciated in FIG. 13, for example, the handle 110 may diverge from the longitudinal axis 112. However, even in the embodiment depicted in FIG. 13, it may be appreciated that the longitudinal axis 112 generally corresponds with (e.g., extends along, adjacent to, or parallel with) a length of the handle 110 even if the entirety of the length of the handle 110 does not extend along the longitudinal axis 112. In any regard, as depicted, the tick remover 100 may be disposed at a first end 102 of the handle 110. In one embodiment, the first end 102 may be provided on an opposite end of the handle 110 from a grooming tool (not shown in FIGS. 1 and 2).

The handle 110 may be grippingly engaged by a user. For example, an outer surface 118 of a handle sidewall 114 may be grippingly engaged by a user when utilizing the tick remover 100 or a grooming tool provided with the handle 110. The sidewall 114 may also define a cross section of the handle 110. The handle 110 may have a cross section that is non-planar along at least a portion of the length of the handle 110. In an embodiment, the handle 110 may have an arcuate cross section. In this regard, at least a portion of the handle 110 may include a curved cross section portion. For example, the cross section of the handle 110 may be elliptical. While the cross section of the handle 110 shown in FIGS. 1 and 2 is substantially circular, it will be understood that the term elliptical may encompass substantially circular cross sections, oval cross sections, or other closed arcuate shapes (e.g., including irregular arcuate shapes). In other embodiments, the cross section of the handle 110 may be polygonal (e.g., triangular, rectilinear, pentagonal, octagonal, etc.).

The sidewall 114 may extend about a hollow space 116 defined interior to the sidewall 114 (e.g., opposite the outer surface 118 of the handle sidewall 114). The hollow space 116 may extend (e.g., longitudinally) along at least a portion of the handle 110. In one embodiment, the hollow space 116 may extend along at least a majority of the length of the handle 110. The hollow space 116 may be accessible through an opening in the sidewall 114. For example, the opening in the sidewall for accessing the hollow space may be provided at the first end 102 of the handle 110.

The sidewall 114 may also include a slot 120. The slot 120 may extend from the first end 102 of the handle 110 in a direction corresponding to the longitudinal axis 112 of the grooming apparatus 10. The slot 120 may be open at the first end 102 to accommodate receipt of a tick or the like into the slot 120. Accordingly, a portion of the tick (e.g., the tick body) may be disposed within the hollow space 116 when the tick is received by the slot 120. In other embodiments, a slot may be provided that extends in a direction corresponding to the longitudinal axis 112, but that is spaced apart from the first end 102 of the handle 110. In this regard, a slot may terminate in an opening in the sidewall 114 (e.g., one spaced apart from the first end 102) that is large enough to receive a tick therethrough so that the tick may be engaged by the slot. In this regard, the slot may not terminate at the first end 102 as depicted in FIGS. 1 and 2.

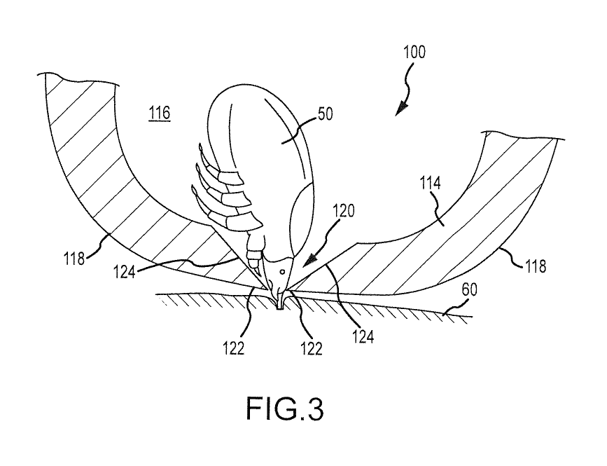

With further reference to FIG. 3, at least a portion of a tick 50 may be received in the slot 120 between opposing edges 122 of the slot 120. The opposing side edges 122 may be tapered along at least a portion of the length of the slot 120 such that the opposing edges 122 converge as the edges 122 extend away from the first end 102. In this regard, as the slot 120 is advanced with respect to the tick 50, the opposing edges 122 may converge so as to contact and engage the tick 50 received therein. Therefore, for differently sized ticks 50, the relative amount of advancement of the slot 120 may be differed to securely engage or contact the differently sized ticks without applying excess pressure to the tick body (e.g., that may lead to squeezing or severing the tick body).

The opposing side edges 122 may also be chamfered. Thus, the opposing side edges 122 may include inwardly facing inclined surfaces 124 that engage the portion of the tick 50 received by the slot 120 as the slot 120 is advanced with respect to the tick 50. The inclined surfaces 124 may converge in a direction toward the exterior surface 118 of the sidewall 114 to define the inwardly facing inclined surfaces 124. As such, the tick 50 may be engaged by the inclined surfaces 124 of the opposing side edges 122 such that upon application of a removal force to the tick 50, the force applied to the tick 50 may be distributed over a surface area of the inclined surfaces 124 of the opposing edges 122 as will be discussed in greater detail below. Furthermore, the opposing side edges 122 may include rounded portions adjacent to the first end. The rounded portions of the opposing side edges 122 at the first end may be near the opening of the slot 120 and may assist in directing a tick toward or into the slot 120 upon advancing the slot 120 relative to the tick 50.

The tick remover 100 may be used to remove a tick 50 from the skin 60 of a host. For instance, the tick remover 100 may be advanced in a direction corresponding to the longitudinal axis 112 of the grooming apparatus 10. A tick 50 that is embedded into the skin 60 of the host may be received between the opposing edges 122 of the slot 120. Once the tick 50 is received in the slot 120, the handle 110 may continue to be advanced until the body of the tick 50 comes into contact with the inclined surfaces 124 of the opposing edges 122. That is, the tick 50 may be located along the converging opposing edges 122 of the slot 120 such that the tick 50 is securely in contact with the inclined surfaces 124, yet is not squeezed or pinched by the opposing edges 122. The handle 110 may then be moved away from the skin 60 of the host in a direction substantially perpendicular to the longitudinal axis 112 to pull the tick 50 from the skin 60. In this regard, it will be appreciated that the inclined surfaces 124 may assist to distribute the force applied to the tick 50 during removal over a larger surface area of the body of the tick 50 along the inclined surfaces 124. This may reduce the likelihood that the mouth parts of the tick 50 break away from the remainder of the body of the tick 50 when being removed from the skin 60.

In that the longitudinal axis 112 of the handle 110 of the grooming apparatus 10 is generally aligned with the slot 120 that may engage the tick 50, the tick remover 100 may provide an ergonomic means for removing the tick 50. That is, a user may generally be operable to grip the handle 110 such that the fingers of the user wrap about the longitudinal axis 112 of the handle 110. Thus, the user may apply a force perpendicular to the longitudinal axis 112 of the handle portion 110 by pulling away in a direction along the length of the user's arm, thus providing an ergonomic means of removing the tick 50. Additionally or alternatively, a portion of the handle 110 may be used as a fulcrum about which the handle 110 may be pivoted. In this regard, the handle 110 may also be leveraged to apply the removal force to the tick 50.

The width of the handle 110 (e.g., corresponding to the diameter of the circular cross section shown in FIGS. 1 and 2) may be not less than about 15 mm and not greater than about 30 mm. For example, the width of the handle 110 may be about 20 mm. Accordingly, the width of the handle 110 may facilitate ergonomic gripping engagement by a user when using a grooming tool and/or the tick remover 100 (e.g., may facilitate gripping of the handle 110 by wrapping four fingers around the handle 110 to secure the handle between the user's fingers and palm). Furthermore, the width of the handle 110 may facilitate a sufficiently large hollow space 116 to accommodate most, if not all sizes of embedded ticks 50, even if engorged with blood.

Furthermore, the thickness of the sidewall 114 may be not less than about 1 mm, 2 mm, or even 3 mm and not greater than about 8 mm, 9 mm, or even 10 mm. For example, in one embodiment, the thickness of the sidewall 114 may be about 3 mm. In this regard, the sidewall 114 may have sufficient thickness to facilitate use of the grooming apparatus 10 during grooming and/or tick removal and withstand fracturing or significant flexing of the grooming apparatus 10 Furthermore, the opposing edges 122 of the slot 120 may be not less than about 3 mm apart and not greater than about 15 mm apart at the widest separation of the opposing edges 122. For example, at the widest separation of the opposing edges 122, the spacing between the edges 122 may be about 8 mm. Accordingly, the opposing edges 122 may be sufficiently spaced apart to accommodate receipt of most, if not all, sizes of ticks. For example, both relatively large and relatively small ticks may be received between the opposing edges 122 by advancing the slot 120 different distances relative to the differently sized ticks. For example, large ticks (e.g., ticks engorged with blood) may be received at or near the opening of the slot 120 where the spacing between the edges 122 may be at its greatest, while small ticks (e.g., recently embedded ticks not yet engorged with blood) may be received at a greater distance along the slot 120 where the spacing between the edges 122 may be less than at the opening of the slot 120. In various embodiments, the slot 120 may extend in a direction corresponding to the longitudinal axis 112 a distance no less than about 5 mm and no greater than about 25 mm. For example, in one embodiment, the slot 120 may extend in a direction corresponding to the longitudinal axis 112 a distance about 15 mm. Accordingly, the slot 120 may extend longitudinally a sufficient distance to facilitate the engagement of differently sized ticks as described above. In a particular embodiment, the handle 110 may extend in the direction corresponding to the longitudinal axis 112 a distance of not less than about 10 cm and not greater than about 15 cm. Further still, in one embodiment an angle defined between opposing edges 122 may be not more than 10 degrees. Therefore, as described above, the converging opposing edges 122 may facilitate engagement of ticks of various sizes by allowing for differently sized ticks to be engaged at different points along the length of the slot 120 between the converging opposing edges 122.

In one embodiment, at least a portion of the grooming apparatus 10 (e.g., at least the handle 110) may be constructed from a polymeric material. For example, the polymeric material may be an ABS plastic. Alternative materials may also be used such as, for example, wood, stainless steel, or other appropriate materials. For example, it is contemplated that the grooming apparatus 10 may be used in the context of grooming, and as such, be exposed to moisture, cleaning chemicals, and other environmental factors attendant to grooming. As such, the material used to construct the grooming apparatus preferably withstands degradation when exposed to such environmental factors associated with grooming.

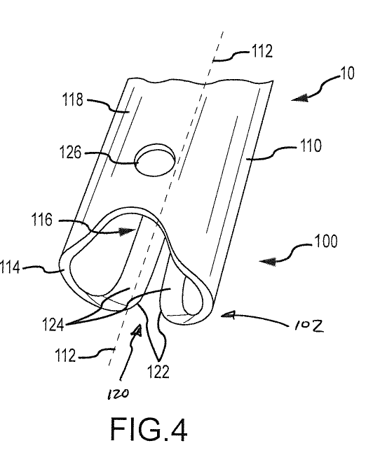

With further reference to FIG. 4, the handle 110 may also be provided with an aperture 126 (e.g., a through hole) extending through the sidewall 114 of the handle portion 110. In this regard, the aperture 126 may be provided for hanging the grooming apparatus 10 or otherwise supporting the grooming apparatus 10 (e.g., when not in use). Additionally or alternatively, the aperture 126 may receive a lanyard (not shown). The aperture 126 may be offset about the handle 110 (e.g., opposite the slot 120) so that even if a lanyard extends through the aperture 126, the operation of the slot 120 when removing a tick 50 may be undisturbed by the lanyard.

Also, as shown in FIG. 4, the handle 110 may include a recessed portion 130 in the sidewall 114 adjacent to the first end 102. The recessed portion 130 may be offset about the handle 110 with respect to the slot 120. For example, as shown, the recessed portion 130 may be on a side of the handle 110 at the first end 102 opposite the slot 120 (e.g., spaced about the handle 110 about 180°). In this regard, the recessed portion 130 may allow a user to view at least the opening of the slot 120 when initially engaging a tick 50 with the slot 120. In this regard, the recessed portion 130 may provide a line of sight of the slot 120 to a user thus facilitating an improved view of the engagement of a tick 50 into the slot 120.

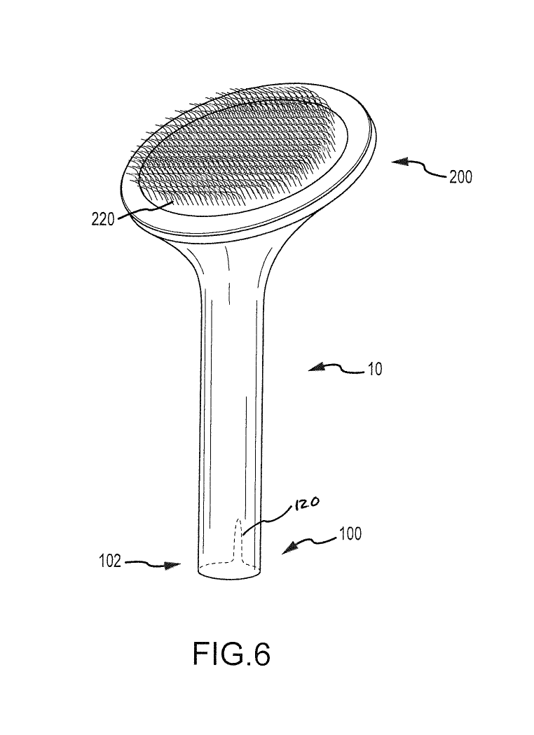

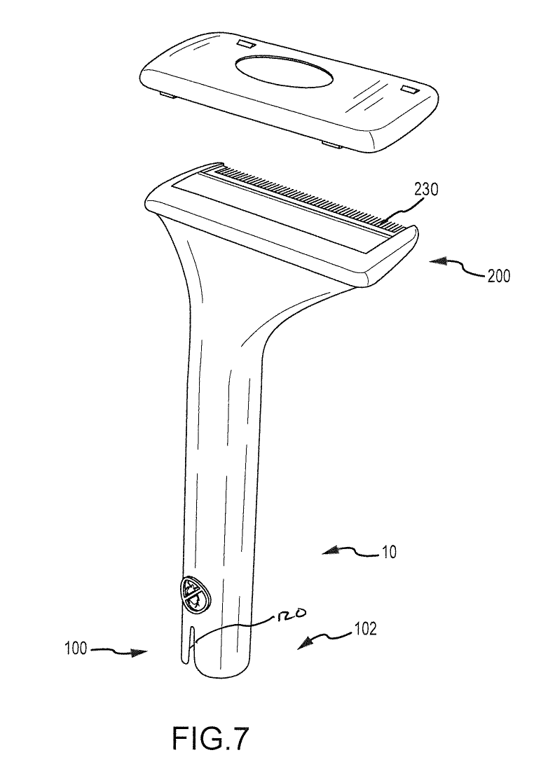

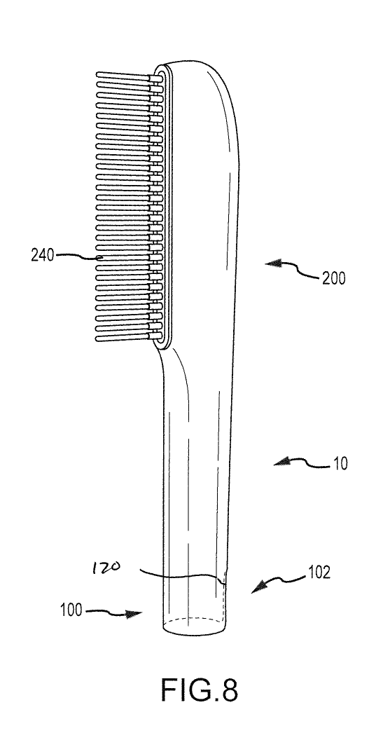

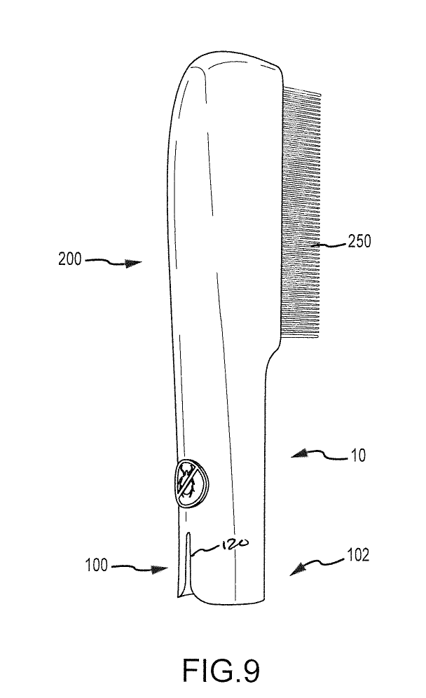

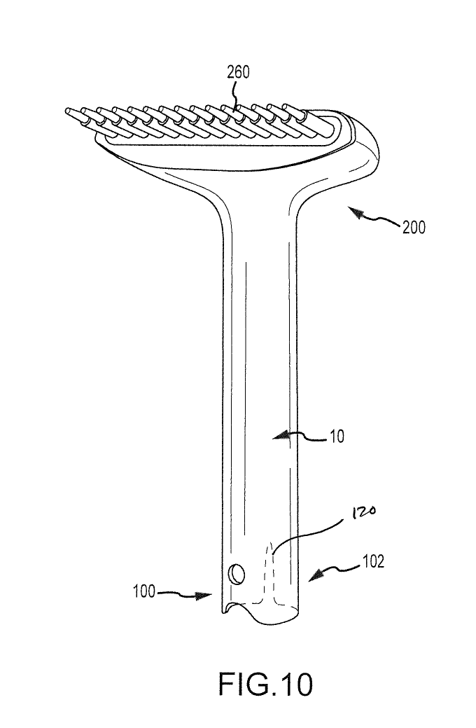

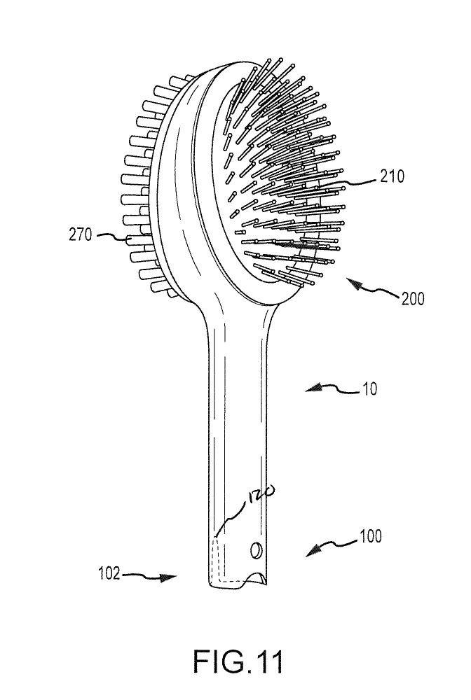

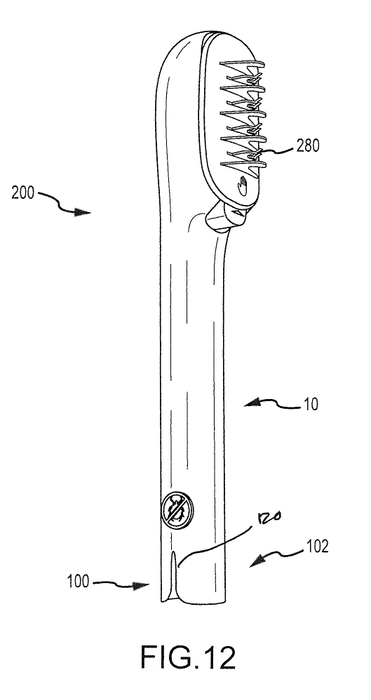

As described briefly above, the tick remover 100 may be provided on the handle 110 of a grooming apparatus 10 opposite that of a grooming tool 200. As shown in FIGS. 5-12, various different embodiments of grooming tools 200 may be provided for various embodiments of a grooming apparatus 10. Grooming tool 200 may be used to generally refer to a grooming tool provided with a grooming apparatus 10. Accordingly, when referring to “grooming tool 200,” any and/or all of the following grooming tools may be referred to collectively or individually. For example, FIG. 5 depicts a pin brush 210, FIG. 6 depicts a slicker brush 220, FIG. 7 depicts a deshedder 230, FIG. 8 depicts a pin comb 240, FIG. 9 depicts a flea comb 250, FIG. 10 depicts an undercoat rake 260, FIG. 11 depicts a multiuse bristle brush 270 and pin brush 210, and FIG. 12 depicts a dematter 280. Other grooming tools known in the art may be provided without limitation.

With respect to any or all of the grooming tools 210-280 shown in FIGS. 5-12, some features may be noted. For example, the grooming tool 200 may be provided integrally with the handle 110. Furthermore, the grooming tool 200 may be provided at an end of the handle 110 opposite the tick remover 100. Accordingly, the longitudinal axis 112 may extend generally between the first end of the handle portion 110 and the grooming tool 200 disposed at the second end of the handle portion 110 opposite the first end 102.

Furthermore, it may be appreciated that in a number of the grooming tools 200 shown in FIGS. 5-12, the slot 120 may be provided on a side of the handle 110 different than a side from which the grooming tool projects. For example, in FIG. 5, the pins of the pin brush 210 generally extend away from the handle 110 a direction opposite from the side on which the slot 120 is provided (e.g., the slot 120 may be offset from the direction in which the grooming tool 200 extends by about 180°). In other embodiments, such as the one shown in FIG. 12, the slot 120 may be offset from the direction in which the grooming tool 200 extends from the handle 110 by about 90°.

In this regard, as discussed above, the slot 120 may be advanced along the skin of the host to engage a tick in the slot 120. As such, the slot 120 may be in close relation (i.e., in direct contact) with the slot of the host. Accordingly, offsetting the slot 120 from the direction in which the grooming tool 200 extends from the handle 110 may reduce the interference of the grooming tool 200 when advancing the slot 120 in close relation to the skin of the host. Furthermore, the embodiment depicted in FIG. 13 may reduce interference of a grooming tool 200 when advancing the handle 110 along the skin of the host. That is, as a portion of the handle 110 may be offset, the grooming tool 200 may be spaced apart from the slot 120 such that the grooming tool 200 may also be offset from the portion of the handle 110 including the slot 120. The offset handle 110 shown in FIG. 13 may also reduce the potential for interference of the grooming tool 200 when advancing the slot 120 in close relation to the skin of the host.

While the invention has been illustrated and described in detail in the drawings and foregoing description, such illustration and description is to be considered as exemplary and not restrictive in character. For example, certain embodiments described hereinabove may be combinable with other described embodiments and/or arranged in other ways (e.g., process elements may be performed in other sequences). Accordingly, it should be understood that only the preferred embodiment and variants thereof have been shown and described and that all changes and modifications that come within the spirit of the invention are desired to be protected.

What is claimed is:

1. A grooming apparatus, comprising:
a handle portion grippingly engageable by a user and having a handle sidewall defining a non-planar cross section, wherein at least a portion of the handle portion defines a longitudinal axis; and
a slot defined in the sidewall and extending in a direction corresponding to the longitudinal axis, wherein the slot is configured to engage at least a portion of a tick between opposing edges of the slot.

2. A grooming apparatus according to claim 1, wherein the handle portion comprises an arcuate cross section at least along a portion thereof.

3. A grooming apparatus according to claim 2, wherein the arcuate cross section is elliptical.

4. A grooming apparatus according to claim 3, wherein the arcuate cross section is substantially circular.

5. A grooming apparatus according to claim 3, wherein the slot extends longitudinally from a first end of the handle portion.

6. A grooming apparatus according to claim 5, wherein the opposing edges of the slot converge as the edges extend away from the first end.

7. A grooming apparatus according to claim 6, wherein the opposing edges are chamfered.

8. A grooming apparatus according to claim 7, wherein the chamfered opposing edges converge toward an exterior surface of the handle sidewall to define inwardly facing inclined surfaces.

9. A grooming apparatus according to claim 6, wherein the handle sidewall defines an opening to a hollow space at the first end, wherein at least a portion of the tick is received in the hollow space when engaged by the slot.

10. A grooming apparatus according to claim 9, wherein the opposing edges are rounded adjacent to the first end.

11. A grooming apparatus according to claim 10, wherein the handle portion comprises a recessed portion offset from the slot adjacent to the first end to provide a line of sight to the slot for a user.

12. A grooming apparatus according to claim 5, further comprising:
a grooming tool disposed at a second end of the handle portion opposite the first end.

13. A grooming apparatus according to claim 5, wherein the slot is offset about the handle portion from a direction in which the grooming tool extends from the handle portion.

14. A grooming apparatus according to claim 5, wherein the slot extends longitudinally from the first end a distance not less than about 5 mm.

15. A grooming apparatus according to claim 14, wherein a width of the arcuate cross section is not less than about 15 mm and not greater than about 30 mm.

16. A grooming apparatus according to claim 15, wherein the thickness of the handle sidewall is not less than about 1 mm thick and not greater than about 10 mm thick.

17. A grooming apparatus according to claim 16, wherein the opposing edges define an angle therebetween of not more than about 10 degrees.

18. A grooming apparatus according to claim 1, wherein the handle portion of the grooming apparatus is constructed from a polymeric material.

19. A grooming apparatus according to claim 18, wherein the polymeric material is ABS plastic.

20. A grooming apparatus, comprising:
a handle portion grippingly engageable by a user and having a handle sidewall defining an arcuate cross section at least at a first end thereof, the handle portion having a second end opposite the first end, wherein a longitudinal axis extends along at least a portion of the handle portion between the first end and the second end;
a grooming tool at a second end; and
a slot defined in the sidewall and extending from the first end in a direction corresponding to the longitudinal axis, wherein the slot is configured to engage at least a portion of a tick between opposing edges of the slot.

21. A grooming apparatus according to claim 20, wherein the arcuate cross section is substantially circular.

22. A grooming apparatus according to claim 20, wherein the grooming tool is selected from the group comprising a pin brush, a bristle brush, a deshedder, a pin comb, a flea comb, a slicker brush, an undercoat rake, a matt breaker, and combinations thereof.

23. A grooming apparatus according to claim 22, wherein the grooming tool is integrally provided with the handle portion.

24. A method for removing an embedded tick from the skin of a host, comprising:
advancing a first end of a handle portion along the surface of the skin of the host in which the tick is embedded, wherein the handle portion is advanced in a direction corresponding to a longitudinal axis of the handle portion;
engaging, in response to the advancing, at least a portion of a tick in a slot defined in a sidewall of the tool handle at the first end;
moving the slot away from the surface of the skin of the host in a direction generally perpendicular to the longitudinal axis of the tool handle; and
removing the tick from the skin in response to the moving.
